(19) United States
(12) Patent Application Publication
(10) Pub. No.: US 2021/0290295 A1
(43) Pub. Date: Sep. 23, 2021

(54) METHODS OF SEPARATING DISSIMILAR MATERIALS WITH ELECTROSURGICAL ELECTRODES AND SYSTEMS AND METHODS INCLUDING SAME

(71) Applicant: Robert James Stanley, II, Cary, NC (US)

(72) Inventor: Robert James Stanley, II, Cary, NC (US)

(21) Appl. No.: 17/337,737

(22) Filed: Jun. 3, 2021

Related U.S. Application Data
(63) Continuation-in-part of application No. PCT/US2019/064104, filed on Dec. 3, 2019.
Application No. PCT/US2019/064104 is a continuation-in-part of application No. 15/830,863, filed on Dec. 4, 2017, now Pat. No. 11,051,872.
(60) Provisional application No. 62/775,036, filed on Dec. 4, 2018.
(60) Provisional application No. 63/162,662, filed on Mar. 18, 2021.

(51) Int. Cl. A61B 18/14

(52) U.S. Cl. CPC A61B 18/1402; A61B 2018/00577; A61B 2018/1407

(57) ABSTRACT

A method for separating at least two dissimilar materials includes providing an electrode having a contact surface, placing the contact surface in contact with at least one material of the at least two dissimilar materials; and sliding the contact surface across and in contact with the at least one material of the at least two dissimilar materials while applying electrosurgical currents via the contact surface to thereby separate the at least two dissimilar materials.

### RELATED APPLICATION INFORMATION

This application is a continuation-in-part of U.S. application Ser. No. 15/830,863 filed on Dec. 4, 2017 and is a continuation-in-part of International Application No. PCT/US2019/064104 filed on Dec. 3, 2019, which claims the benefit of U.S. Application Ser. No. 62/775,036, filed Dec. 4, 2018, and this application claims the benefit of U.S. Application Ser. No. 63/162,662, filed Mar. 18, 2021, the disclosure of each of which is incorporated herein by reference in their entirety.

### FIELD OF THE INVENTION

The present invention relates to electrosurgical apparatus and methods and, more particularly, to electrosurgical electrodes and electrosurgical apparatus and methods including the same.

### BACKGROUND

Electrosurgery is a common procedure for dentists, doctors, and veterinarians. Electrosurgical handpieces are commercially available that will accommodate a wide variety of electrode shapes and sizes, such as needles, blades, scalpels, balls and wire loops. Also, multi-function electrodes are available.

The electrodes can be used in many surgical procedures in which a conventional scalpel is employed, mainly for general cutting procedures. An electrosurgical scalpel electrode has the advantage of providing electrosurgical currents at the sharp edge of the scalpel, which assist in cutting tissue while at the same time providing a coagulation effect. Another known shape is the ball electrode, which is a spherical ball on the end of an electrode shank used for coagulation.

While these various shaped electrodes are suitable for their intended purposes of cutting and coagulation, occasions arise from time to time when these electrodes are pressed into service to ablate tissue. In this situation, the known electrodes typically fall short of a desired outcome.

### SUMMARY

According to embodiments of the invention, a method for treating tissue of a subject includes: providing an electrode having a contact surface, optionally wherein the contact surface has a curved profile; placing the contact surface in contact with tissue of the subject; and sliding the contact surface across and in contact with the tissue while applying electrosurgical currents to the tissue via the contact surface to thereby remove a portion of the tissue from the subject and form a treated area of the tissue.

According to further embodiments of the invention, a method for performing electrosurgery on tissue of a subject comprises: providing an electrosurgical apparatus including: an electrode including a contact surface and a socket defined in the electrode; and a wiping insert removably mounted in the socket; contacting the contact surface with the tissue while applying electrosurgical currents to the tissue via the contact surface to thereby electrosurgically treat the tissue; and thereafter wiping the tissue using the wiping insert in the socket.

According to some embodiments of the invention, a method for removing one or more epithelial layers from a tissue of a subject includes: providing an electrode having a contact surface, optionally wherein the contact surface has a curved profile; placing the contact surface in contact with a first epithelial layer of the tissue of the subject; and sliding the contact surface across and in contact with the first epithelial layer while applying electrosurgical currents to the first epithelial layer via the contact surface to thereby remove the one or more epithelial layers from the tissue of the subject.

According to some embodiments of the invention, a method for separating at least two dissimilar materials includes providing an electrode having a contact surface, placing the contact surface in contact with at least one material of the at least two dissimilar materials; and sliding the contact surface across and in contact with the at least one material of the at least two dissimilar materials while applying electrosurgical currents via the contact surface to thereby separate the at least two dissimilar materials.

According to embodiments of the invention, a method for separating at least two dissimilar tissues of a subject includes: providing an electrode having a contact surface; placing the contact surface in contact with at least one tissue of the at least two dissimilar tissues; and sliding the contact surface across and in contact with the at least one tissue while applying electrosurgical currents to the via the contact surface to thereby separate the at least two dissimilar tissues and/or remove at least one dissimilar tissue from the other tissue(s).

According to some embodiments of the invention, a method for separating at least two dissimilar materials of an article includes: providing an electrode having a contact surface; placing the contact surface in contact with a surface of the article; and sliding the contact surface across and in contact with the surface of the article while applying electrosurgical currents to the surface of the article via the contact surface to thereby separate the at least two dissimilar materials of the article.

In some embodiments of the invention, the contact surface of the electrode has a curved or otherwise rounded profile.

It is noted that aspects of the invention described with respect to one embodiment, may be incorporated in a different embodiment although not specifically described relative thereto. That is, all embodiments and/or features of any embodiment can be combined in any way and/or combination. These and other objects and/or aspects of the present invention are explained in detail in the specification set forth below.

### BRIEF DRAWING DESCRIPTION

The accompanying figures are included to provide a further understanding of the present invention, and are incorporated in and constitute a part of this specification. The drawings illustrate some embodiments of the present invention and, together with the description, serve to explain principles of the present invention.

FIG. 1 is a schematic, perspective view of an electrosurgical system according to embodiments of the invention.

FIG. 2 is a fragmentary, exploded, perspective view of an electrode according to embodiments of the invention and forming a part of the system of FIG. 1.

FIG. 3 is a side view of the electrode of FIG. 2.

FIG. 4 is a top view of the electrode of FIG. 2.

FIG. 5 is an enlarged, fragmentary, top view of the electrode of FIG. 2.

FIG. 6 is a fragmentary, cross-sectional view of the electrode of FIG. 2 taken along the line 6-6 of FIG. 5.

FIG. 7 is a cross-sectional view of the electrode of FIG. 2 taken along the line 7-7 of FIG. 5.

FIG. 8 is a fragmentary, top view of the system of FIG. 1 being used to execute a brushing step on a subject's tissue to form a treated area of tissue.

FIG. 9 is a fragmentary, cross-sectional view of the system and subject tissue of FIG. 8 taken along the line 9-9 of FIG. 8.

FIG. 10 is a fragmentary, top view of the system of FIG. 1 being used to execute a brushing step on a subject's gum tissue to form a treated area of gum tissue.

FIG. 11 is a fragmentary, cross-sectional view of the system and subject tissue of FIG. 10 taken along the line 11-11 of FIG. 10.

FIG. 12 is a histological image of a pigmented tissue prior to treatment.

FIG. 13 is a histological image of the tissue of FIG. 12 on the day the procedure following treatment, which shows that all epithelium and pigmentation was removed from the treated tissue.

FIG. 14 is a histological image of the tissue of FIG. 13 months after treatment, which shows that the tissue re-epithelized and that the re-epithelized tissue was not pigmented tissue.

### DETAILED DESCRIPTION OF EMBODIMENTS OF THE INVENTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present. Like numbers refer to like elements throughout.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

Well-known functions or constructions may not be described in detail for brevity and/or clarity.

As used herein the expression “and/or” includes any and all combinations of one or more of the associated listed items.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the terms “increase,” “increases,” “increased,” “increasing,” “enhance,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more.

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40%, 45%, 50%, 55%, 60%, 65%, 70%, 75%, 80%, 85%, 90%, 95%, 97%, or 100%.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “monolithic” means an object that is a single, unitary piece formed or composed of a material without joints or seams.

As used herein, “operator” may include a physician, veterinarian, dentist, nurse, dental hygienist, or other clinician, and/or a scientist, researcher, or member of the general public, for example. As used herein, “operator” may include two or more people in collaboration.

As used herein, “dissimilar materials” refers to any two or more materials that have significantly different impedance values, or for DC circuits, different resistance values. In some embodiments, the impedance and/or resistance value of one of the two or more materials is greater than the other material(s) by more than 10%, 15%, 20%, 25%, 30%, 35%, 40%, 45%, 50%, 55%, 60%, 65%, 70%, 75%, 80%, 85%, 90%, 95%, 100% or more. Examples of materials include organic materials, including human or animal tissues and other biopolymers; plastics and other synthetic polymers; metals; wood; fibers, and/or any other suitable material. In some embodiments, different types of dissimilar materials may be separated (e.g., plastic and metal, tissue and fibers, etc.).

As used herein, “ablate” or “ablation” refers to removal of a material by mechanical means (e.g., by electrosurgery) and/or by the erosion of a surface, e.g., by heating caused by an electrode of an embodiment of the present invention.

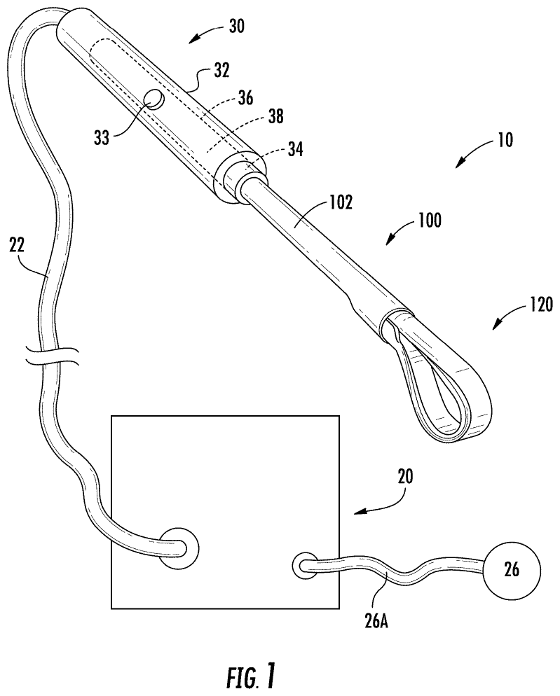

With reference to FIGS. 1-11, an electrosurgical system 10 according to some embodiments of the invention is shown therein. With reference to FIG. 1, the system 10 includes a brush electrode 100 according to some embodiments of the invention. The system 10 further includes an electrosurgical apparatus 20, a handpiece 30, and an electrically insulated electrical cable 22 operatively connecting the handpiece 20 to the electrosurgical apparatus 20.

In accordance with methods of the invention, the electrosurgical system 10 and the brush electrode 100 can be used to separate two or more (e.g., 2, 3, 4, or more) dissimilar materials. In some embodiments, methods for separating at least two dissimilar materials include providing an electrode having a contact surface; placing the contact surface in contact with at least one material of the at least two dissimilar materials; and applying electrosurgical currents to the at least one material via the contact surface while the contact surface is in contact with the at least one material to thereby separate the two dissimilar materials. In some embodiments, the contact surface is straight and/or flat. In some embodiments, the contact surface has a curved profile. In some embodiments, methods for separating at least two dissimilar materials include providing an electrode having a contact surface, optionally wherein the contact surface has a curved profile; placing the contact surface in contact with at least one material of the at least two dissimilar materials; and sliding the contact surface across and in contact with the at least one material while applying electrosurgical currents via the contact surface to thereby separate the two dissimilar materials. In some embodiments, one or more of the dissimilar materials has no or minimal damage after the separation.

In some embodiments, a method, electrosurgical system 10 and brush electrode 100 can be used to separate two dissimilar tissues of a subject. In some embodiments, a method, electrosurgical system 10 and/or brush electrode 100 of the present invention may be used on and/or to treat all or a portion of a subject's tissues. For example, in some embodiments, two or more dissimilar tissues may be present in a human or animal and it may be desirable to separate such materials. In some embodiments, epithelium tissue is separated from a dissimilar material such as, e.g., loose connective tissue (e.g., oral mucosa or tissue of the alimentary canal) or dense connective tissue (e.g., gingiva). In some embodiments, periosteum may be separated from a dissimilar material, such as, e.g., loose connective tissue, glandular tissue, adipose tissue, or bone. Other examples include, but are not limited to, separating epithelium from dense connective tissue (e.g., gingiva), separating epithelium from loose connective tissue (e.g., oral mucosa, alimentary canal), separating loose connective tissue from the periosteum (e.g., in a periodontal surgery), separating adipose tissue from connective tissue or periosteum, separating glandular tissue from the connective tissue or periosteum, separating epidermis from the dermis, separating dermis from subcutaneous tissue (e.g., fat, blood vessels, connective tissue), separating muscle from tendons, separating tendons from bones, and the like.

As such, the methods provided herein may be used on any portion of the subject's body, including in the mouth, hands, feet, arms, legs, head, pelvis, chest, neck, and the like. In some embodiments, the separation of tissues may be useful in transplantation of tissues, either from a live person or a cadaver (collectively, a “donor”) to a recipient. In some embodiments, one or more of the dissimilar tissues has no or minimal damage after the separation from the other tissue(s). In some embodiments, a method of the present invention may be used to remove a subject's tonsil and the method may remove the tonsil and have no or minimal damage to surrounding structures. In some embodiments, a method of the present invention may be used for tumor removal for a subject (e.g., removal of a tumor that a normal scalpel would remove and likely provide damage to the surrounding and/or healthy tissue (e.g., brain tissue) and may have no or minimal damage to surrounding structures.

According to some embodiments of the invention, a method for separating at least two dissimilar materials of an article includes: providing an electrode having a contact surface, optionally wherein the contact surface has a curved profile; placing the contact surface in contact with a surface of the article; and sliding the contact surface across and in contact with the surface of the article while applying electrosurgical currents to the surface of the article via the contact surface to thereby separate the at least two dissimilar materials of the article.

Example articles include, but are not limited to, electronic articles, metal, wood, fiber, plastic and other polymeric articles, and/or organic articles. In some embodiments, the two dissimilar materials comprise two dissimilar plastics and/or polymers. In some embodiments, the organic article is a subject or tissue therefrom. In some embodiments, the organic article is a plant or a product therefrom (e.g., fruit, seed, etc.). For example, in some embodiments, a method of the present invention may be used to separate two dissimilar layers of a fruit or vegetable (e.g., a method of the present invention may be used to peel produce (e.g., a potato, apple and/or orange)).

The surface of the article may comprise one of the two dissimilar materials and, responsive to contacting the surface of the article with the electrode 100, the other of the two dissimilar materials may be exposed. In some embodiments, the other of the two dissimilar materials may be isolated from the article to thereby provide an isolated material.

In some embodiments, a conductive layer is present between the two dissimilar materials and thereby may separate at least a portion of the two dissimilar materials (e.g., tissues). The conductive layer may directly contact (i.e., no intervening layer or component) or indirectly contact one or more of the two dissimilar materials of the article. In some embodiments, at least a portion of the conductive layer separately contacts each of the two dissimilar materials. The conductive layer may be part of the structure of the article, or it may be added, applied to, injected, or otherwise introduced into the interface (collectively referred to as “introduced”) between the at least to dissimilar materials.

In some embodiments, at least one conductive substance may be present in and/or on at least one of the dissimilar materials. As such, in some embodiments, a conductive substance may be added, injected, applied to, or otherwise introduced (collectively referred to as “introduced”) in at least one of the two dissimilar materials. Such a conductive substance may change the impedance of the material and thus may create a larger difference in impedance values between the two dissimilar materials. In some embodiments, a first material that is not dissimilar, as defined herein, to a second material may be rendered dissimilar by the introduction of the conductive substance. The conductive substance may vary depending on the application but in some embodiments, the conductive substance is a saline solution.

In some embodiment of the invention, at least one of the materials may be modified prior to placing the contact surface of the electrode in contact with the at least one material of the at least two dissimilar materials. In some embodiments, the hydration level of at least one of the at least two dissimilar materials may affect the impedance between the at least two dissimilar materials. As such, increasing or decreasing hydration and/or water content of a material may be done prior to performing and/or during a method of the invention. For example, if the dissimilar materials are part of a subject (e.g., a human or animal), the subject may undergo a particular food or diet regimen in advance in order to provide a change in the impedance of at least one of the subject's tissues. In some embodiments, the hydration of the subject may affect the impedance of certain tissues. As such, a subject may increase (hydrate) or decrease (dehydrate) water intake prior to performing a method of the invention. Further, other types of materials may be modified to alter the impedance, for example, by supplementation (e.g., ingestion, injection, etc.), heating, cooling, processing, and the like. In some embodiments, a saline solution may be used to modify (e.g., increase or decrease) the impedance difference between two dissimilar materials. For example, a saline solution or another biologically appropriate conductive medium may be provided (e.g., added, injected, etc.) into a space between two materials (e.g., two tissues) and may become a conductive path for radiofrequency energy and thereby allow for separation of the two materials that normally would not have a large enough impedance difference in order to separate the two materials.

In some embodiments, a method of the present invention vaporizes and ablates one of the two dissimilar materials, optionally leaving the other of the two dissimilar materials substantially unaltered.

Embodiments, properties, and features of the present invention are described further below with regard to the article being a subject for simplicity. Properties and/or features of the method for separating two dissimilar materials from an article may be as described further below and duplicate discussion thereof may be omitted herein.

“Tissue” as used herein includes any tissue of a subject including, but not limited to, a subject's skin, mucosa, and/or organ(s). In some embodiments, a method, electrosurgical system 10 and/or brush electrode 100 of the present invention may be used on and/or to treat a subject's epithelial tissue. In some embodiments, a method, electrosurgical system 10 and/or brush electrode 100 of the present invention may be used on and/or to treat a soft tissue of a subject such as, for example, all or a portion of the subject's gum. In some embodiments, a method, electrosurgical system 10 and/or brush electrode 100 of the present invention may be used on and/or to treat a subject's skin such as, for example, all or a portion of the subject's skin covering their arm(s), leg(s), face, and/or torso. In some embodiments, a method, electrosurgical system 10 and/or brush electrode 100 of the present invention may be used on and/or to treat a subject's mucous membrane (including a mucous membrane in a body cavity) such as, for example, all or a portion of the subject's vagina and/or oral mucosa. In some embodiments, a method, electrosurgical system 10 and/or brush electrode 100 of the present invention may be used for cosmetic purposes such as, for example, to reduce the appearance of and/or remove at least a portion of a scar, tattoo, hyperpigmented region, and/or hypopigmented region on the tissue of a subject.

Subjects include, but are not limited to, mammalian subjects and avian subjects. Mammals of the present invention include, but are not limited to, humans and animals such as, e.g., canines, felines, bovines, caprines, equines, ovines, porcines, rodents (e.g. rats and mice), lagomorphs, non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments, the subject is a mammal. In some embodiments, the subject is a human. Human subjects include both males and females of all ages including fetal, neonatal, infant, juvenile, adolescent, adult, and geriatric subjects as well as pregnant subjects. In some embodiments, the subject is a human adolescent and/or adult. The subject (e.g., human) may be alive or deceased (i.e., a cadaver or portions thereof) and the term human, animal, or subject includes both living and deceased human, animals, and subjects, respectively, unless otherwise indicated.

Illustrative avians according to the present invention include, but are not limited to, chickens, ducks, turkeys, geese, quail, pheasant, ratites (e.g., ostrich) and domesticated birds (e.g., parrots and canaries).

A method, electrosurgical system 10 and/or brush electrode 100 of the present invention may also be used on and/or to treat animal subjects (e.g., mice, rats, dogs, cats, livestock and horses) for veterinary purposes and/or research purposes. In some embodiments, the subject is a dog.

In some embodiments, the subject is “in need of” or “in need thereof” a method of the present invention, for example, the subject is in an at-risk population (e.g. the subject may be at-risk for basal cell carcinoma), the subject has findings typically associated with a condition that may be suitable for a method of the present invention (e.g., the subject has a birthmark, mole, dark or blotchy gum tissue, etc.), is suspected to have a condition that may be suitable for a method of the present invention, and/or the subject has a condition that may be suitable for a method of the present invention. In some embodiments, a subject has a skin lesion and/or mucosa lesion that may be treated with a method of the present invention.

A skin lesion and/or mucosal lesion on a subject may be treated and/or removed according to embodiments of the present invention. Example skin lesions include, but are not limited to, macules (e.g., freckles), moles, birthmarks (e.g, pigmented birthmarks and vascular birthmarks), warts, sores, plaques, pustules, and/or actinic keratosis. Example mucosal lesions include, but are not limited to, leukoplakia, erythroplakia, erythroleukoplakia, leukoedema, herpes lesions, warts, and/or frictional keratosis. In some embodiments, the skin lesion and/or mucosal lesion is benign. In some embodiments, the skin lesion and/or mucosal lesion is premalignant. In some embodiments, the skin lesion and/or mucosal lesion is cancerous.

According to some embodiments, a method, electrosurgical system 10 and/or brush electrode 100 of the present invention may be used to remove leukoplakia, erythroplakia, and/or erythroleukoplakia on a subject. In some embodiments, a method, electrosurgical system 10 and/or brush electrode 100 of the present invention may be used to treat a subject that has hairy tongue.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with a tissue is achieved and/or there is a delay in the progression of the symptom. In some embodiments, the severity or degree of a lesion on a tissue of a subject may be reduced compared to the severity or degree of the lesion in the absence of a method of the present invention. For example, a method of the present invention may reduce the area on the skin of a subject that exhibits a darker shade of pigmentation than surrounding skin compared to the area of darker pigmentation in the absence of the method.

According to some embodiments, a method of the present invention may prevent a condition (e.g., a skin and/or mucosa condition) from developing. For example, in some embodiments, a method of the present invention may remove a skin lesion and/or mucosal lesion, which may prevent the skin lesion and/or mucosal lesion from becoming cancerous. The terms “prevent,” “preventing” and “prevention” (and grammatical variations thereof) refer to avoidance, reduction and/or delay of the onset of a condition and/or a reduction in the severity of a condition relative to what would occur in the absence of a method of the present invention. The prevention can be complete, e.g., the total absence of the condition. The prevention can also be partial, such that the occurrence of the condition in the subject and/or the severity of onset is less than what would occur in the absence of a method of the present invention.

As discussed herein, the system 10 and the electrode 100 can be used to separate and/or remove one or more (e.g., 1, 2, 3, 4, 5, 6, 7 or more) dissimilar layers of an article such as, e.g., one or more layers of a tissue for a subject. In some embodiments, the system 10 and the electrode 100 are used to separate and/or remove one or more layers of a subject's skin and/or mucosal tissue. The one or more layers of the tissue may be one or more epithelial layers. One, two, three, four, five or more layers of epithelial cells may be removed from a subject. For example, the electrosurgical system 10 and/or brush electrode 100 may be used to separate and/or remove all or a portion of one or more layers of the epidermis. According to some embodiments of the present invention, all or a portion of the stratum basale, stratum spinosum, stratum granulosum, and/or stratum corneum in a given area (e.g., a 1 cm×1 cm area) of tissue for a subject are removed. The given area for a tissue is a region of the tissue that is all or a portion of the tissue and for which a surface of the tissue in the given area is contacted with the electrode 100. In some embodiments, about 5%, 10%, 15%, 20%, 25%, 30%, 35%, 40%, 45%, 50%, 55%, 60%, 65%, 70%, 75%, 80%, 85%, 90%, 95%, 97%, or 100% of the stratum basale, stratum spinosum, stratum granulosum, and/or stratum corneum in a given area (e.g., a 1 cm×1 cm area) of tissue for a subject are removed from the given area (with 100% being the total area of the given area). The system 10 and the electrode 100 may be used in a method of the present invention to separate and/or remove all or a portion of the epithelium from underlying connective tissue. In some embodiments, about 50%, 55%, 60%, 65%, 70%, 75%, 80%, 85%, 90%, 95%, 97%, or 100% of connective tissue and/or dermis in a given area of tissue for a subject remain intact and/or unaltered after a method of the present invention. Basal cells present in the epidermis (e.g., in the stratum basale) of a subject may stop the separation and/or removal of tissue below the basal cells.

In some embodiments, a method of the present invention removes a material (e.g., tissue) in a given area to a depth in the range from about 0.01, 0.25, or 0.5 mm to about 0.75, 1, 2, 3, 4, or 5 mm. According to some embodiments, the removed material (e.g., tissue) depth is substantially uniform (i.e., within ±20%) across the width of the treated area of the article. In some embodiments, the material or tissue below any material removed according to a method of the present invention (e.g., connective tissue and/or dermis) may have no damage or minimal damage. In some embodiments, damage to a material below any material removed according to a method of the present invention (e.g., connective tissue and/or dermis) may have a depth of about 0.01 mm to about 0.15 mm. In some embodiments, a method of the present invention may remove a layer of material (e.g., tissue) and leave the underlying topography substantially intact. Thus, in some embodiments, a method of the present invention may remove a layer of material that has a varying depth. For example, in some embodiments, a method of the present invention may remove the epidermis and the rete pegs of the epidermis and leave the dermal papillae intact such that the pattern of the dermal papillae is substantially intact, that is the peaks and valleys of the dermal papillae are maintained.

Material (e.g., tissue) in a given area may be contacted one or more (e.g., 1, 2, 3, 4, 5, 10, 15, 20, or more) times with the electrode 100 during a treatment session. For example, the electrode 100 may be contacted to (e.g., brushed on) a given area of tissue one or more times until a desired level of tissue separation and/or removal has been achieved. This may include contacting a first tissue region with the electrode 100 to provide an exposed second tissue region and contacting the second tissue region with the electrode 100 to provide an exposed third tissue region and so on until a desired level of tissue separation and/or removal has been achieved.

In some embodiments, the system 10 and the electrode 100 can be used to electrosurgically vaporize and thereby ablate tissue by contacting the electrode 100 with the tissue while applying RF current from the electrosurgical apparatus 20 to the tissue through the electrode 100. In some embodiments, an energized contact surface of the electrode is contacted to (e.g., touches) a surface of a tissue such that the electrode contact surface ablates a portion or all of the tissue contacted with the electrode contact surface and forms a treated area of the tissue. In some embodiments, an energized contact surface of the electrode is brushed or slid over a surface of a tissue such that the electrode contact surface ablates a portion or all of the tissue contacted with the electrode contact surface and forms a treated area of the tissue. In some embodiments, the treated area of tissue is relatively broad and a substantially uniform depth of the tissue is ablated across the width of the treated area. In some embodiments, the area of the treated area is substantially the same (e.g., ±20%) or the same as that of the area of the given area. In some embodiments, the ablated tissue is exposed surface tissue. In some embodiments, the ablated tissue comprises a first layer of tissue (e.g., a first epithelial layer) that was in contact with the electrode contact surface and optionally one or more additional tissue layers (e.g., one or more epithelial layers). In some embodiments, the ablated tissue is skin tissue. In some embodiments, the ablated tissue is mucosal tissue (e.g., gum tissue).

The electrode 100 may also be used to cut or scrape tissue without the application of the RF current. For example, the electrode 100 can be used to scrape away tissue that has been desiccated or coagulated by the aforementioned ablating step. In some embodiments, the electrode 100 may be used to cut a treated area (e.g., a treated area comprising connective tissue and/or dermis) optionally to isolate (e.g., remove) the treated area from the subject.

In some embodiments, the electrosurgical system 10 and the brush electrode 100 can also be used to electrosurgically cut, scrape, cauterize, coagulate, and/or desiccate tissue by contacting the electrode 100 with the tissue while applying RF current from the electrosurgical apparatus 20 to the tissue through the electrode 100.

The skin of a subject contacted with the electrode 100 may be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the subject's total body surface area (TBSA). In some embodiments, about 50% or more of a subject's TBSA is contacted with the electrode 100 during a single treatment session. In some embodiments, less than about 0.5% or 1% of a subject's TBSA is contacted with the electrode 100 during a single treatment session.

According to some embodiments, a method of the present invention comprises removing epidermis from a subject, the method comprising contacting the electrode 100 with the epidermis while applying RF current from the electrosurgical apparatus 20 to the epidermis through the electrode 100. Any epidermis desired to be removed may be contacted with the electrode 100. The method may result in separating and/or removing the epidermis from dermis, thereby providing exposed dermis. The exposed dermis may be used to obtain and/or isolate acellular dermis from the subject. The method may provide a quicker and/or more efficient production of acellular dermis. The acellular dermis may be used in burn victims, breast reconstruction, tendon repair, hernia repair, and/or to rebuild turbinates. The brush electrode 100 may be selected to have a given width to aid in providing a uniform and/or quick removal of the epidermis. In some embodiments, a method of preparing acellular dermis is provided and the method is devoid of chemicals to remove the epidermis from the subject. The subject in a method of preparing acellular dermis may be deceased and optionally an isolated portion (e.g., an organ, arm, etc.) of the deceased subject may be used to prepare acellular dermis.

A method of the present invention may comprise contacting the electrode 100 with a skin lesion and/or mucosa lesion on a subject while applying RF current from the electrosurgical apparatus 20 to the skin lesion and/or mucosa lesion through the electrode 100. A portion or all of the skin lesion and/or mucosa lesion may be removed and/or the area of the skin lesion and/or mucosa lesion may be reduced by about 5%, 10%, 15%, 20%, 25%, 30%, 35%, 40%, 45%, 50%, 55%, 60%, 65%, 70%, 75%, 80%, 85%, 90%, 95%, 97%, or 100%. In some embodiments, a method of the present invention may be used to treat and/or prevent skin cancer such as, e.g., basal cell carcinoma and squamous cell carcinoma. A method of the present invention may remove a cancerous skin and/or mucosal lesion before it metastasizes. In some embodiments, the system 10 and the electrode 100 are used to remove a basal cell carcinoma before it invades the underling connective tissue. In some embodiments, the system 10 and the electrode 100 are used to reduce the appearance of and/or remove a mole and/or a birthmark on a subject.

A method of the present invention may comprise modifying (i.e., increasing or decreasing) the pigmentation in a tissue (e.g., skin and/or mucosa membrane) of a subject, the method comprising contacting the electrode 100 with the tissue while applying RF current from the electrosurgical apparatus 20 to the tissue through the electrode 100. An area of tissue may be darker and/or lighter in color than tissue surrounding the area of tissue. For example, a mole on the skin of a subject may be more darkly pigmented than the skin surrounding the mole. A method of modifying the pigmentation in the tissue (e.g., skin and/or mucosa membrane) of a subject may modify the pigmentation in a given area of tissue to be closer to the pigmentation color of the tissue surrounding the given area of tissue. In some embodiments, the subject may have and/or be suspected to have vitiligo and a method of the present invention may be used to treat and/or prevent vitiligo.

A method of the present invention may reduce the appearance of a skin lesion, mucosal lesion, scar, tattoo, hyperpigmented region, and/or hypopigmented region on the tissue of a subject by about 5%, 10%, 15%, 20%, 25%, 30%, 35%, 40%, 45%, 50%, 55%, 60%, 65%, 70%, 75%, 80%, 85%, 90%, 95%, 97%, or 100% compared to the appearance of the skin lesion, mucosal lesion, scar, tattoo, hyperpigmented region, and/or hypopigmented region in the absence of the method. Reduction in the appearance of the skin lesion, mucosal lesion, scar, tattoo, hyperpigmented region, and/or hypopigmented region may be determined using methods known to those of skill in the art including visual determinations.

According to some embodiments, a method of the present invention may be used to obtain tissue from a donor (e.g., a live subject or a cadaver) and/or to prepare tissue for grafting to a recipient. The method of obtaining tissue from a donor may comprise contacting the electrode 100 with the tissue while applying RF current from the electrosurgical apparatus 20 to the tissue through the electrode 100, thereby providing a treated area of tissue and isolating the treated area of tissue from the donor. Isolating the treated area of tissue from the donor may comprise removing (e.g., cutting) the treated area to separate it from surrounding tissue. In some embodiments, a recipient is transplanted with a portion of the tissue isolated from the donor. The portion of the tissue isolated from the donor may be washed and/or contacted with medication(s) to prepare it for transplantation. In some embodiments, the tissue isolated from the donor and/or transplanted to the recipient comprises connective tissue and/or dermis. In some embodiments, tissue from the roof of a subject's mouth (donor site) is taken modified by the removal of the epithelium, and then placed in an intraoral site of a recipient. Using current surgical techniques (prior to those of the present invention) it can be difficult to visually differentiate epithelium from the underlining connective tissue. The current separation process can involve using a blade to cut off the top layer of epithelium. This can be a crude technique with poor precision and accuracy due to the lack of a clearly visible delineation of the tissues and the clinician's hand skills with the blade. The result may be too much epithelium removed or inadvertently leaving epithelium tissue on the desired graft. A method of the present invention may improve the precision and/or accuracy in obtaining donor tissue. In some embodiments, a method of the present invention may provide complete removal of epithelium from a donor site and may preserve at least about 80%, 85%, 90%, 95%, 99%, of the underlying connective tissue.

The electrosurgical apparatus 20 may be any suitable electrosurgical apparatus. According to some embodiments, the electrosurgical apparatus 20 is a radiofrequency (RF) radiosurgical energy source operable to selectively generate and deliver alternating polarity electrical current (hereinafter, referred to as an RF generator). In some embodiments, the electrosurgical apparatus is an ultra-high frequency RF generator. Suitable electrosurgical RF generators may include the RADIOSURGE 3™ electrosurgical unit available from Ellman International, Inc. of Hewlett, N.Y. In some embodiments, the apparatus 20 provides current to the electrode 100 at a high operating frequency (in some embodiments, in the range of 2 to 4 MHz and, in some embodiments, about 3.8-4 MHz). In some embodiments, the apparatus 20 provides power to the electrode 100 in a range of about 1 Watt to about 150 Watts. A method of the present invention may contact the electrode 100 to a tissue with the apparatus 20 supplying to the electrode 100 about 1 Watt to about 30 Watts, about 5 Watts to about 50 Watts, about 10 Watts to about 25 Watts, about 50 Watts to about 150 Watts, or about 100 Watts to about 150 Watts. In some embodiments, a method of the present invention comprises contacting a tissue with the electrode 100 at a low power (e.g., at about 1 Watt) and increasing the power until the desired result is achieved (e.g., a desired amount of tissue is removed). The electrosurgical apparatus 20 may include one or more switches that enable an operator to selectively turn the supplied RF current on and off. For example, the apparatus 20 may include a switch 33 (e.g., a button switch) on the handpiece 30 and a redundant foot operable switch (not shown).

The system 10 may further include a dispersive electrode 26 operatively electrically connected to the electrosurgical unit 20 or to electrical ground by an electrical cable 26A. In use of the system 10, the dispersive electrode 26 is mounted on and in electrical contact with the subject's body and spaced apart from the surgical region. The dispersive electrode 26 operates to disperse the RF current and thereby prevent unintended injury to the tissue underlying the tissue intended to be treated using the electrode 100. The electric current oscillates between the electrode 100 and the dispersive electrode 26 with the subject interposed between the electrodes 100, 26.

The handpiece 30 includes a handle 32 and a locking mechanism 34 such as a collet. A bore 36 is located in the handle 32 and contains an electrical contact 38. The electrical contact 38 is electrically connected to the cable 22. The electrical contact 38 may be a tubular member.

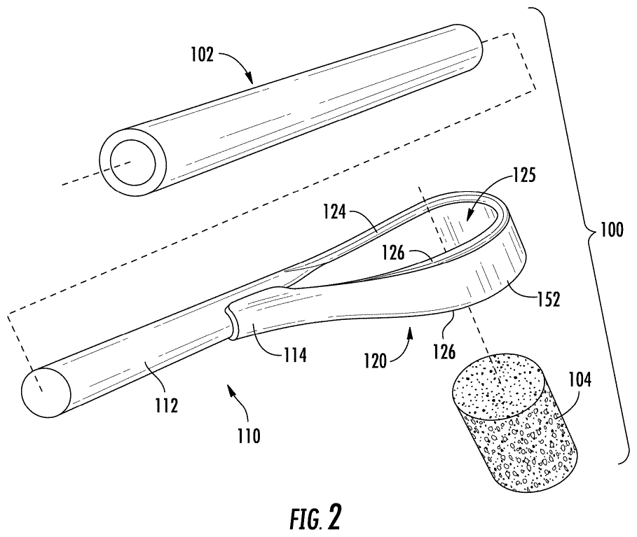

The brush electrode 100 includes an electrode member 110 and an insulation sleeve 102. The electrode 100 may further include a supplemental wiping insert 104 (FIGS. 2-4). In some embodiments, the insert 104 is a pliable, absorbent member and, in some embodiments, is a sponge or gauze.

The electrode member 110 is formed of an electrically conductive material or materials. In some embodiments, the electrode member 110 is formed of metal. Suitable metals may include brass, gold, silver, stainless steel, or molybdenum. In some embodiments, the electrode member 110 is a unitary body. In some embodiments, the electrode member 110 is monolithic. In some embodiments, the electrode member 110 is a spheroid or ovoid shape, optionally a solid, hollow, or substantially hollow spheroid or ovoid shape. In some embodiments, the electrode 100 is a different enclosed shape having rounded edges (e.g., lightbulb-shaped). Such an enclose shape may be solid, hollow, or substantially hollow. The electrode 100 may also be flat with rounded or curved sides that facilitate movement across the surface of a material. In some embodiments, the electrode 100 is in the form of a “stamp” that does not move across the material but is pressed onto a material.

The insulation sleeve 102 is formed of an electrically insulating material or materials. The insulation sleeve 102 may be a preformed component or a coating. In some embodiments, the insulation sleeve 102 is formed of a polymeric insulating material. Suitable materials may include heat shrinkable thermoplastic. The insulation sleeve 102 surrounds a portion of the electrode member 110 to prevent the electrode 100 from inadvertently burning the subject. The electrode 110 may also have a coating on a portion of all of the electrode 110 such that the coating facilitates movement of the electrode 110 across one or more of the dissimilar materials. In some embodiments, such coatings may include non-stick and/or low surface energy polymers (e.g., polytetrafluoroethylene).

The electrode member 110 includes a shank 112, a working portion (or active end portion) 120, and a tail portion 114. The shank 112 is a straight rod disposed in the insulation sleeve 102, except for a connector portion 112A that extends proximally from the insulation sleeve 102. The shank 112 may have an outer diameter of about 1/16 inch.

In use, the electrode 100 is inserted into the bore 36 of the handpiece to electrically connect the working portion 120 to the contact 38. The contact 38 is in turn electrically connected to the apparatus 20 via the cable 22. The insulation sleeve 102 electrically insulates the portion of the electrode member 110 extending between the handpiece 30 and the working end 120.

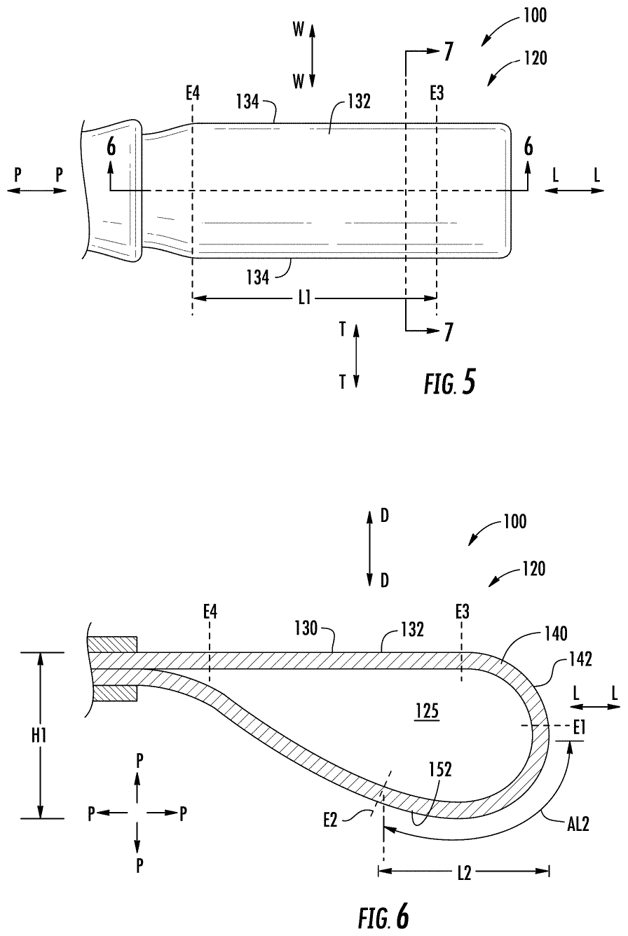

With reference to FIGS. 5-7, the working portion 120 has a primary or longitudinal axis L-L, a widthwise or first lateral axis W-W that is perpendicular to the longitudinal axis L-L, and a depth-wise or second lateral axis D-D that is perpendicular to the longitudinal axis L-L and perpendicular to the first lateral axis W-W. The working portion 120 extends from a distal end 122A (FIG. 3) to an opposing proximal end 122B. The working portion 120 has an outer or top side 122C and an opposing subject facing or bottom side 122D.

The working portion 120 includes a thin band or strip 124 having opposed lateral edges 126. The strip 124 is configured to form or define a loop 128. In some embodiments and as shown, the loop 128 is generally tear drop-shaped in a cross-sectional profile plane P-P (FIGS. 5 and 6), the plane P-P being parallel to the longitudinal axis L-L and parallel to the depth-wise axis D-D. In some embodiments and as shown, the loop 128 is generally tear drop-shaped in all planes parallel to the profile plane from lateral edge 126 to lateral edge 126.

The working portion 120 may be formed by any suitable technique. In some embodiments, the working portion 120 is formed by bending a metal part. In some embodiments, the strip 124 and the tail portion 114 are formed by flattening a portion of a metal rod (e.g., by drawing) to a prescribed width and then bending the strip 124 back into the shape of the loop 128.

In some embodiments, the lateral edges 126 or portions thereof are sharp. In some embodiments, one or both of the edges 126 is sharpened (e.g., by cutting, machining, extrusion, or casting) to a sharp edge. In some embodiments, the lateral edges 126 have substantially the same thickness as the remainder of the strip 124.

The working portion 120 includes a top wall 130, a bottom wall 150 and a transition wall 140 connecting the top wall 130 to the bottom wall 150. The working portion 120 is hollow so that the walls 130, 140, 150 collectively form a socket 125. The socket 125 extends laterally (substantially parallel to the axis W-W) and terminates at opposed openings 125A.

The top wall 130 includes a planar outer surface 132. The planar outer surface 132 extends lengthwise from a distal end E3 to a proximal end E4, and laterally from a first lateral edge 134 to an opposing, parallel lateral edge 134. In some embodiments, the top wall lateral edges 134 are sections of the strip lateral edges 126. According to some embodiments, the outer surface 132 is substantially continuous and substantially planar from end E3 to end E4 and edge 134 to edge 134.

In some embodiments, the planar outer surface 132 has a length L1 (FIG. 5) in the range of from about 6.5 mm to about 11.5 mm.

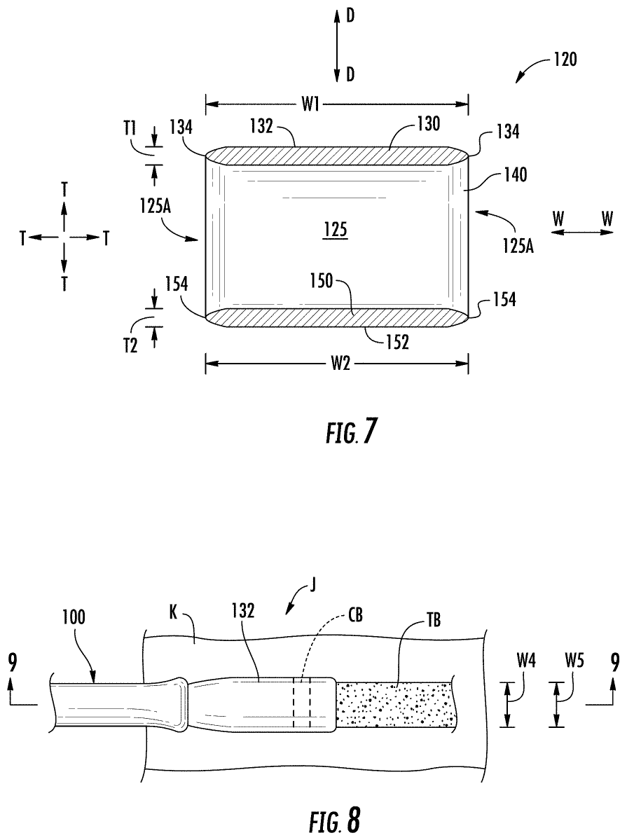

In some embodiments, the planar outer surface 132 has a width W1 (FIG. 7) in the range of from about 1 mm to about 4 mm.

In some embodiments, the planar outer surface 132 has an area in the range of from about 6.5 mm² to about 48 mm².

In some embodiments, the top wall 130 has a thickness T1 (FIG. 7) in the range of from about 0.5 mm to about 1.0 mm.

The bottom wall 150 is partially lobe-shaped. The bottom wall 150 has an outer contact surface 152. As discussed in more detail below, the bottom wall 150 and the contact surface 152 may each have a rounded, curved or arcuate longitudinal profile, and a linear or flat widthwise profile.

The contact surface 152 extends lengthwise from a distal end E1 to a proximal end E2, and laterally from a first lateral edge 154 to an opposing, parallel lateral edge 154. In some embodiments, the contact surface lateral edges 154 are sections of the strip lateral edges 126. According to some embodiments, the contact surface 152 is substantially continuous from end E1 to end E2 and edge 154 to edge 154.

The contact surface 152 has a curved or arcuate shape and defines a curved contact surface profile in the profile plane P-P. In some embodiments and as shown, the contact surface 152 has the curved profile P in substantially all planes parallel to the profile plane P-P from lateral edge 154 to lateral edge 154 and from the end E1 to the end E2.

The curved profile P has an arc length AL2 (FIG. 6) extending from end E1 to end E2. The profile P is convex relative to the space below the bottom wall 150 and, when used to ablate by brushing, is convex relative to the surface of the tissue being treated. According to some embodiments, there are no sharp corners, sharp edges, hard angles or sharp transitions in the curve of the profile P. According to some embodiments, the profile P follows a smooth, continuous curve. In some embodiments, the profile P is a non-uniform curve.

In some embodiments, the profile P is or includes a truncated elliptical shape. In some embodiments, the profile P is or includes a partial cylindrical shape. In some embodiments, the profile P has a half heart curve shape.

In some embodiments, the minimum arc radius of the profile P is at least 1.5 mm. In some embodiments, the minimum arc radius of the profile P is in the range of from about 1.5 mm to about 2.0 mm.

In some embodiments and as shown, the contact surface 152 is curved in the longitudinal direction and flat in the lateral direction from end E1 to E2. That is, the contact surface 152 is flat or planar in a cross-sectional transverse plane T-T (FIGS. 5 and 7) that is parallel to the widthwise axis W-W and the depth-wise axis D-D. In some embodiments, the contact surface 152 is flat or planar in substantially all cross-sectional transverse planes parallel to cross-sectional transverse plane T-T from lateral edge 154 to lateral edge 154 and from the end E1 to the end E2.

According to some embodiments and as shown, there are no openings in the contact surface 152.

In some embodiments, the contact surface 152 has a linear length L2 (FIG. 6) in the range of from about 2 mm to about 4 mm.

In some embodiments, the contact surface 152 has an arc length AL2 (FIG. 6) in the range of from about 3 mm to about 5 mm.

In some embodiments, the contact surface 152 has a width W2 (FIG. 7) that is at least 1 mm and, in some embodiments, is in the range of from about 1 mm to about 4 mm.

In some embodiments, the contact surface 152 has an area in the range of from about 1 mm² to about 8 mm².

In some embodiments, the bottom wall 150 has a thickness T2 (FIG. 7) in the range of from about 0.5 mm to about 1 mm.

The transition wall 140 connects the top wall 120 to the bottom wall 150 and extends between the planar outer surface 132 and the curved contact surface 152. In some embodiments, the outer surface 142 of the transition wall 140 has a curved or arcuate shape in the profile plane P-P. In some embodiments and as shown, the outer surface 142 has a curved profile in substantially all planes parallel to the profile plane P from lateral edge 126 to lateral edge 126. In some embodiments and as shown, the curved profile P of the outer surface 142 is convex. According to some embodiments, the curved profile P of the outer surface 142 is without sharp edges or transitions and follows a smooth, continuous curve. In some embodiments, the profile is or includes a truncated elliptical shape. In some embodiments, the profile is or includes a partial cylindrical shape.

In some embodiments and as shown, the outer surface 142 is curved in the longitudinal direction and flat in the lateral direction as discussed above with regard to the contact surface 152. That is, the outer surface 142 is flat or planar in the cross-sectional transverse plane T-T and, in some embodiments, is flat or planar in substantially all cross-sectional transverse planes parallel to cross-sectional transverse plane T-T from lateral edge 126 to lateral edge 126.

In some embodiments, the height distance HI (FIG. 6) between the top wall surface 132 and the lowest point of the contact surface 152 is in the range of from about 2.5 mm to about 3.5 mm.

In some embodiments, the socket 125 has a volume in the range of from about 7.1 mm³ to about 50.25 mm³.

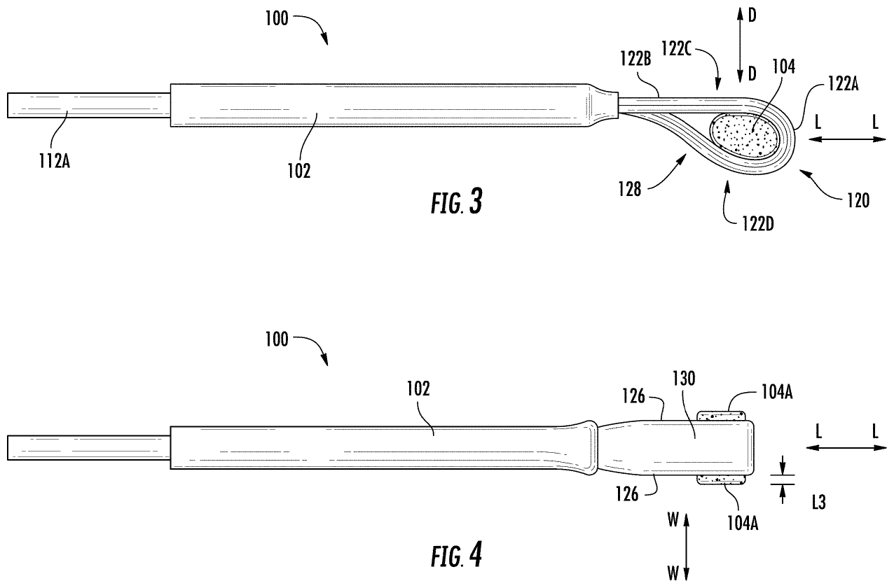

In some embodiments, the supplemental wiping insert 104 is a sponge or gauze. The supplemental wiping insert 104 may be disposable. In some embodiments, the insert 104 is formed of an electrically nonconductive material. In some embodiments, the insert 104 (e.g., sponge or gauze) is sized such that it can be inserted into the socket 125 with extension portions 104A of the insert 104 projecting outwardly through the openings 125A and beyond one or both of the lateral edges 126 a distance L3 (FIG. 4). In some embodiments, the distance L3 is in the range of from about 0 mm to about 1 mm.

The electrode 100 may be formed by bending or otherwise forming a metal component into the shape of the electrode member 110 as described above. The insulation sleeve 102 is mounted around the shank 112 and the terminal end of the tail portion 114. For example, the insulation sleeve 102 may be heat-shrunk about the electrode member 110. Covering the tail portion 114 in this manner prevents the free end of the tail portion 114 from being exposed, where it may interfere with use of the electrode 100 (e.g., by catching on the subject or objects in the surgical field or the surgical table like gauze).

The system 10 and the electrode 100 may be used as discussed above and further below in accordance with embodiments of the invention. Depending on the intended use or application, the electrode 100 may have a shape (e.g., a curved or flat contact surface) and/or size suitable for the intended use or application. Thus, methods of the present invention may be executed with electrodes having different configurations, and a method described herein may utilize an electrode having a different or the same configuration as an electrode for another method.

The system 10 and the electrode 100 may be used to ablate tissue by vaporization in accordance with some method embodiments and the electrode 100 is especially well-suited to vaporize and ablate tissue in a controlled manner. The system 10 and the electrode 100 may be used to ablate skin tissue. The electrode 100 may be used to conduct cosmetic ablative procedures on skin tissue. The system 10 and the electrode 100 may also be used to scrape, cut, cauterize, coagulate, and/or desiccate tissue of a subject.

The electrode 100 may be used in different operational modes including a brushing mode, a cutting mode, a scraping mode, and a wiping mode.

In the brushing mode the electrode 100 is used to execute a brushing step, wherein an operator drags or slides the contact surface 152 of the working portion 120 in contact with and across an outer surface of tissue of a subject. As the electrode brushes across the tissue, it ablates a layer of the tissue.

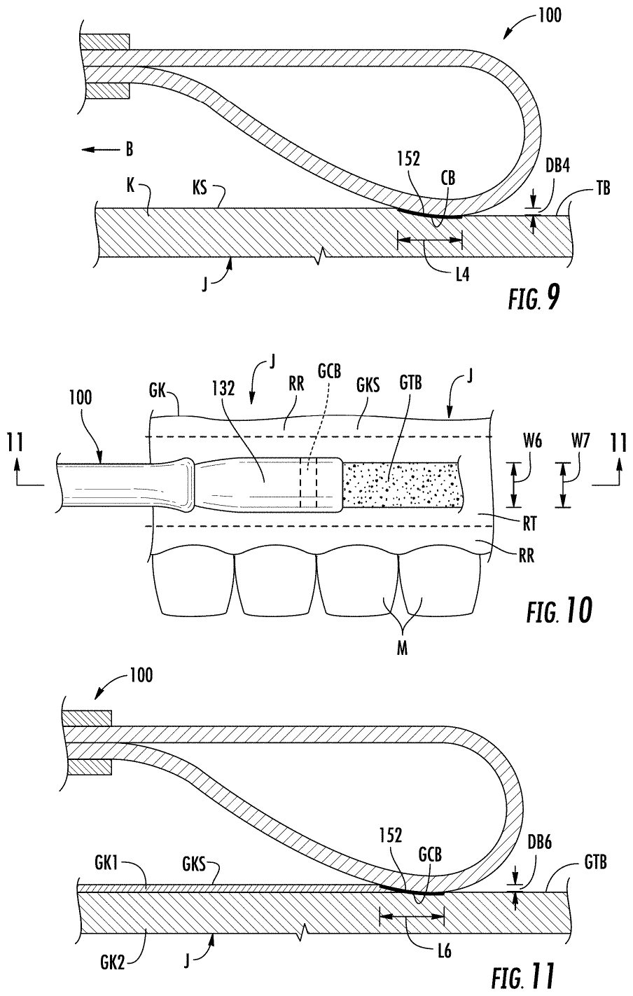

For example, FIGS. 8 and 9 illustrate the working portion 120 being slid or brushed across a surface KS of a tissue K of a subject J. The working portion 120 is placed in contact with the tissue surface K such that a contact band CB is formed. The contact band CB is the region of tissue K that is in contact with the contact surface 152 at any given time. The dimensions of the working portion 120 may be selected to correspond to the dimensions of the area to be contacted with the working portion 120, optionally to reduce the number of times the electrode 100 is contacted to the tissue of the subject.

The contact surface 152 is maintained in contact with the tissue surface K while the contact surface 152 is displaced or wiped across the tissue K in a direction B. Simultaneously with the brushing movement, the electrosurgical apparatus 20 is operated to deliver RF energy to the contact surface 152 as discussed above. Displacing the electrode 100 in this manner progressively advances the contact band CB to thereby create a broad swath or treated area TB of treated tissue. In some embodiments, the electrode vaporizes and ablates the tissue in the contact band CB, so that the brushing step vaporizes and ablates a broad band of a layer of tissue and the treated area TB is tissue from which overlying tissue has been vaporized and ablated. In some embodiments, a method of the present invention may deliver a RF energy that increases over time until a desired RF energy is reached and/or a desired level of tissue removal is achieved.

The broad width of the contact band CB facilitates quicker surgical times. In some embodiments, substantially the entire area of the tissue within the treated area TB is ablated by the brush stroke.

In some embodiments, the depth DB4 (FIG. 9) of ablation across the width of the treated area TB is substantially uniform.

In some embodiments, the procedure includes executing a plurality of light pressure brush strokes over the tissue to create a controlled and uniform depth of tissue removal by ablation. The electrode 100 can be brushed in both the direction B and the opposite direction.

The broad line of contact (i.e., the contact band CB) distributes the energy from the apparatus 20 over a greater area, which may decrease undesired damage to collateral tissues.

In some embodiments, the CB has a width W4 (FIG. 8; transverse to the brush stroke direction B) that is greater that its length L4 (FIG. 9; generally parallel to the direction B). In some embodiments, the ratio of the width W4 to the length L4 is in the range of from about 1 mm to about 2 mm.

The length L4 of the contact band CB may be a function of the pressure applied to the tissue by the electrode 100 and/or the location of the contact band CB on the profile of the contact surface 152. Thus, the length L4 of the contact band CB may vary between a thin line to a thick line or band extending across the width of the treated area TB. In some embodiments, the length L4 is in the range of from about 1 mm to about 3 mm, in some embodiments is in the range of from about 1 mm to about 2 mm, and, in some embodiments, is in the range of from about 2 mm to about 3 mm.

In some embodiments, the width W4 of the contact band CB is in the range of from about 1 mm to about 4 mm. In some embodiments, the width W4 is substantially the same as the width W2 of the contact surface 152. In some embodiments, the width W4 is substantially the same as the width W5 (FIG. 8) of the treated area TB.

As discussed above, in some embodiments, throughout the brushing step or stroke the electrode contact surface 152 applies only a light load or pressure onto the tissue to be ablated.

According to some embodiments, the electrode 100 does not cut tissue during the brushing step.

The contact band CB is tangential to the curve of the profile P. The smooth, continuous shape of the contact surface 152 and the contact band CB facilitates minimal drag resistance between the electrode 100 and the tissue for quick, precise execution of procedures (especially ablative procedures).

The broad width W4 of the contact band CB and the geometry of the contact surface 152 (i.e., the smooth, gradual curvature of the curved profile P) reduce the depth of entry or embedding of the electrode 100 into the tissue for a given pressure of working portion 120 onto the tissue K. This enables the operator to more easily and accurately modulate or control the depth of entry into the tissue. As a result, the operator can more effectively and reliably prevent the working portion 120 from cutting the tissue when cutting is not desired. The operator can better control the depth of treatment of the tissue by ohmic heating. The operator can better limit ablation of the tissue to a shallow depth, if desired.

During the brushing step, the operator can gauge the position of one of the lateral edges 126 of the contact surface 152 by visually observing and monitoring the opposite parallel lateral edge 126. Each of the parallel lateral edges 126 can thereby serve as a lateral field spatial reference for the other to enable the operator to better ascertain and track the position of the contact surface lateral edges 154, and thus the contact band CB, relative to the subject. This feature can be particularly advantageous when one of the lateral edges 126 of the working portion 120 is obscured (e.g., by the subject's anatomy or equipment). The parallel lateral edges 126 allow the operator to have spatial awareness and navigate demarcated regions to prevent collateral tissue damage.

During the brushing step, the operator can also gauge the depth-wise position of the contact surface 152 by visually observing and monitoring the planar top wall surface 132, which is disposed a fixed distance above and overlying the contact surface 152 when the top wall surface 132 is substantially parallel to the tissue being ablated. The surface 132 can thereby serve as a depth field spatial reference to enable the operator to better ascertain and track the depth position of the contact surface 152, and thus the contact band CB, relative to the subject. This feature can be particularly advantageous when the operator wants to carefully limit the depth of entry of the contact surface 152 into the tissue.

In the cutting mode, the electrode 100 is used to execute a cutting step wherein it cuts (and, in some cases, coagulates) tissue of the subject. Either or both of the lateral edges 126 can be used to cut the tissue. The electrode 100 can be used to electrosurgically cut tissue with the electrosurgical apparatus 20 operated to deliver RF energy to the contact surface 152 as discussed above. The electrode 100 can be used to cut tissue while non-energized.

In the scraping mode, the electrode 100 is used to execute a tissue scraping step. Either or both of the lateral edges 126 can be used to scrape the tissue. In some embodiments, the electrode 100 is used to scrape tissue with the electrode non-energized (i.e., without the electrosurgical apparatus 20 operated to deliver RF energy to the contact surface 152 as discussed above). However, in other embodiments, the electrode 100 may be energized during scraping.

The scraping step may be employed to remove tissue residue that has been volatized, desiccated, coagulated or otherwise treated by a brushing step or cutting step as described above. The provision of scraping edges 126 on the brushing electrode 100 relieves the operator of the need to exchange the brush electrode 100 for another instrument and can thereby increase the safety of the subject by reducing surgical time.

In the wiping mode, the electrode 100 is used to execute a wiping step to remove tissue from the surgical region. In the wiping mode, the insert 104 is mounted in the socket 125 as shown in FIGS. 3 and 4. The extensions 104A are then used to wipe material from the tissue of the subject. Typically, the electrode 100 is used to wipe tissue with the electrode 100 non-energized.

In some embodiments, the wiping step is employed to remove tissue residue that has been volatized, desiccated, coagulated, cut or scraped by a brushing step, cutting step or scraping step as described above. The provision of the wiping insert 104 on the brushing electrode 100 relieves the operator of the need to exchange the brush electrode 100 for another instrument, and can thereby increase the safety of the subject by reducing surgical time.

In some embodiments, the system 10 includes a plurality of electrodes 100 of different shapes and/or sizes to permit the operator to customize the system to the treatment, subject, or surgical step at hand.

In some embodiments, the socket 125 may be omitted or filled with an electrically conductive or nonconductive material. For example, the working portion 120 may be solid (i.e., void free).

Portions of the electrode 100 may be bendable to customize the shape or angle of the electrode 100 to permit the operator to customize the system to the treatment, subject, or surgical step at hand. For example, the working portion 120 and/or the shank 112 may be malleable. The electrode 100 may be bent into or supplied in different configurations to provide access to challenging spaces. Methods of using the system and electrode may include bending the electrode prior to use or between steps.

The system 10 and the electrode 100 may be used as discussed above and further below in accordance with embodiments of the invention to execute dental gum treatments. The system 10 and the electrode 100 may be used to ablate gum tissue by vaporization. In some embodiments, the electrode 100 is used to ablate a layer offending gum tissue to effect a cosmetic treatment.

With reference to FIGS. 10 and 11, the electrode 100 is used in the brushing mode to vaporize and ablate a surface layer GK1 of a gum tissue GK of a subject J. In the brushing mode, the operator drags or slides the contact surface 152 of the working portion 120 in contact with and across an outer surface of the gum tissue GK of the subject in the manner described above with reference to FIGS. 8 and 9.

For example, FIGS. 10 and 11 illustrate the working portion 120 being slid or brushed across a surface GKS of the gum tissue GK. The subject's teeth M are also illustrated. The working portion 120 is placed in contact with the tissue surface GKS such that a contact band CB is formed. The contact surface 152 is maintained in contact with the tissue surface GKS while the contact surface 152 is displaced or wiped across the gum tissue GK in a direction B. Simultaneously with the brushing movement, the electrosurgical apparatus 20 is operated to deliver RF energy to the contact surface 152 as discussed above. In this manner, the contact band GCB is progressively advanced to thereby create a broad swath or treated area GTB of treated gum tissue. The electrode 100 may vaporizes and ablate at least the surface gum tissue in the contact band GCB, so that the brushing step vaporizes and ablates a broad area of gum tissue and the treated area GTB is gum tissue from which overlying tissue has been vaporized and ablated. Tissue in the treated area GTB may be killed but not ablated, as well.

According to some embodiments, with reference to FIG. 11, the brushing step ablates and/or kills an epithelium layer GK1 of the subject's gum tissue while leaving an underlying connective tissue layer GK2 substantially unaffected, undamaged or minimally damaged by ablation or heat. In some embodiments, the depth of ablation DB6 (FIG. 11) created by the brushing step is less than 0.01 mm and, in some embodiments, is in the range of from about 0.01, 0.25, or 0.5 mm to about 0.75, 1, 2, 3, 4, or 5 mm. Any damage to the underlying connective tissue layer GK2 may have a depth of about 0.01 mm to about 0.15 mm. According to some embodiments, the depth of ablation DB6 is substantially uniform across the width of the treated area GTB. In some embodiments, the depth of ablation DB6 follows the underlying surface topography of the underlying connective tissue layer GK2 (e.g., the method removes the epithelial extensions (i.e., rete pegs) and leaves the topography of the projections of the dermis (i.e., dermal papillae). In some embodiments, the width W6 (FIG. 10) of the contact band GCB is greater than the length L6 (FIG. 11) of the contact band GCB. In some embodiments, the ratio of the width W6 to the length L6 is in the range of from about 1 mm to about 2 mm.

As discussed above, the length L4 of the contact band GCB may be a function of the pressure applied to the tissue. In some embodiments, the length L4 is in the range of from about 0.5 mm to about 2 mm, in some embodiments is in the range of from about 0.5 mm to about 1 mm, and, in some embodiments, is in the range of from about 1 mm to about 2 mm.

In some embodiments, the width W6 (FIG. 10) of the contact band GCB is in the range of from about 1 mm to about 4 mm. In some embodiments, the width W6 is substantially the same as the width W2 of the contact surface 152. In some embodiments, the width W6 is substantially the same as the width W7 (FIG. 10) of the treated area GTB.

In some embodiments, the electrode 100 is used to treat gingival hyperplasia.

In some embodiments, the electrode 100 is used to treat racial pigmentation. The electrode may be used to treat both gingival hyperplasia and racial pigmentation in the same procedure.

As discussed above, in some embodiments, throughout the brushing step or stroke the electrode contact surface 152 applied only a light load or pressure onto the gum tissue to be ablated.

The smooth curved profile of the contact surface 152 enables the electrode 100 to slide smoothly across the tissue as it ablates. According to some embodiments, the electrode 100 does not cut gum tissue during the brushing step.

The broad width W4 of the contact band CB and the geometry of the contact surface 152 (i.e., the smooth, gradual curvature of the curved profile P) reduce the depth of entry or embedding of the electrode 100 into the tissue for a given pressure of working portion 120 onto the gum tissue GK. This enables the operator to more easily and accurately modulate or control the depth of entry into the gum tissue. As a result, the operator can more effectively and reliably prevent the working portion 120 from cutting the gum tissue when cutting is not desired. The operator can better control the depth of treatment of the gum tissue by ohmic heating. The operator can better limit ablation of the gum tissue to a shallow depth, if desired.

During the brush step, the operator can gauge the position of one of the lateral edges 126 of the contact surface 152 by visually observing and monitoring the opposing parallel lateral edge 126 as described above. In particular, an exposed parallel lateral edge 126 can be used as a lateral field spatial reference for the other to enable the operator to better ascertain and track the position of an obscured lateral edge relative to a restricted region RR (demarcated in FIG. 10 by dashed lines). The restricted region RR may be gum tissue that should not be ablated (“non-offending tissue”) such as tissue proximate the lip line. The lateral edge 126 of the electrode proximate the restricted region may be obscured by the subject's lip.

During the brushing step, the operator can also gauge the depthwise position of the contact surface 152 by visually observing and monitoring the planar top wall surface 132, which is disposed a fixed distance above and overlying the contact surface 152 when the top wall surface 132 is substantially parallel to the tissue being ablated, as discussed above. The surface 132 can thereby serve as a depth field spatial reference to enable the operator to better ascertain and track the depth position of the contact surface 152 into the subject's gum tissue GK. This feature can be particularly advantageous when the operator wants to carefully limit the depth of entry of the contact surface 152 into the gum tissue.

Following the brushing step, the operator may cut and/or scrape gum tissue from the treated area using the cutting mode and/or the scraping mode as discussed above. Gum tissue may be cut or scraped from the treated region using either or both of the lateral edges 126. The electrode 100 can be used to cut and/or scrape tissue either with or without the electrosurgical apparatus 20 operated to deliver RF energy to the contact surface 152 as discussed above. In some embodiments, the electrode 100 is used to scrape gum tissue without the electrosurgical apparatus 20 operated to deliver RF energy to the contact surface 152 in order to avoid unintended damage to the underlying tissue. In some embodiments, in the scraping step, a lateral edge 126 is used to scrape away or remove tissue that has been coagulated by the ablation step, such as collagen from the treated gum tissue that has become gelatinized.

In some embodiments, the system 10 and electrode 100 are operated in a wiping mode to remove gum tissue from the surgical region using the insert 104 (or other insert) mounted in the socket 125, as discussed above. The extensions 104A may be used to wipe ablated, coagulated or otherwise loosened gum tissue from the subject. Typically, the electrode 100 is used to wipe tissue with the electrode 100 non-energized. In particular, the wiping step may be employed to remove gum tissue that has been desiccated, coagulated, cut or scraped by the gum brushing step and the gum scraping step as described above. In some embodiments, in the wiping step, the insert 104 is used to wipe away or remove tissue that has been coagulated by the ablation step, such as collagen from the treated gum tissue that has become gelatinized.

The operator may make multiple brush strokes with the electrode 100 as needed to ablate a desired region of the gum tissue. For example, the operator may continue to brush a target region RT (demarcated by dashed lines in FIG. 10) of tissue (“offending tissue”) bounded by restricted regions RR of tissue (“non-offending tissue”) until most or substantially all of the target region RT has been ablated as described. Scraping and wiping steps as described above may be executed between brush strokes to remove residual tissue in order to clear the surgical field for further ablation or inspection. The operator may alternate between the brushing, scraping and wiping steps as needed to progressively ablate and clear the target region RT.

For example, in procedures according to some embodiments, the operator uses the brushing, scraping and wiping steps and techniques described above as follows:

a) The operator prepares the system 10, including installing the electrode 100 in the handpiece 30;

b) With the electrode 100 energized, the operator brushes tissue in the target region RT with the contact surface 152 to thereby remove (optionally vaporize and ablate) the tissue contacted with the electrode 100. The operator may take care not to unintentionally contact tissue in the restricted regions RR by monitoring the position of the working portion 120 using visual observation of the lateral edges 126, as discussed above. The operator may control the depth of tissue removal using visual observation of the planar surface 132, as discussed above;

c) With the electrode 100 de-energized, the operator scrapes residual tissue (e.g., volatilized residual tissue) from the removal process (e.g., coagulated collagen or other tissue) from the target region RT using the edges 126, as described above;

d) With the electrode 100 de-energized, the operator inserts the wiping insert 104 into the socket 125 and wipes residual tissue from the ablative process from the target region RT using the end sections 104A, as described above. The wiped residual tissue may include residual tissue loosened by the scraping step;

e) The procedure may include executing a plurality of brush strokes over the tissue to create a controlled and uniform depth of tissue removal. The operator can brush the electrode 100 in both the direction B and the opposite direction. The operator may repeat steps a), b) and c) as needed to progressively ablate and clear the target region RT. The operator may remove the dirtied wiping insert 104 from the electrode and replace it with a new, clean wiping insert 104 to use in subsequent wiping steps.

The electrode 100 can be used to conduct cosmetic ablative procedures on the skin and/or mucosal membranes (e.g., gums). According to some embodiments, the electrode 100 and the electrosurgical gum treatment methods described above are used to treat gingival hyperplasia. In this condition, the subject has an excess of gingival tissue growth and discoloration of the gums. The brushing step is used to ablate the epithelium layer (corresponding to layer GK1), and the scraping and wiping steps are used to remove the remaining residual tissue from the ablation.

The electrode 100 can be used to conduct cosmetic ablative procedures on the gums. According to some embodiments, the electrode 100 and the electrosurgical gum treatment methods described above are used to treat racial pigmentation. In this condition, the subject has uneven or undesirably dark coloration of the gums. The brushing step is used to ablate the epithelium layer (corresponding to layer GK1), and the scraping and wiping steps are used to remove the remaining residual tissue from the ablation.

The system 10 may further include a family or set of a plurality of brush electrodes 100 of different widths. The operator can then select the brush electrode or electrodes from the set having the desired width(s) for the intended procedure. For example, the width of electrode may be chosen dependent on the constraints of the surgery.

Electrodes according to embodiments of the present invention can overcome various problems that may otherwise be encountered when electrodes of known shapes (e.g., spherical ball electrodes) are pressed into service to ablate tissue. As compared to such known electrodes, electrodes of the present invention can provide shorter surgical times, more complete removal of offending tissue, more even depth of tissue removal, reduced heat transfer (which may cause undesirable tissue damage) to the tissue, improved visualization of the surgical field, and an overall better aesthetic outcome.

Systems and methods as disclosed herein can enable an operator (e.g., a physician) to efficiently and precisely perform ablative treatments with less time, less cost, fewer follow-up procedures, less collateral tissue damage, and less bleeding compared to known electrosurgery apparatus and procedures. For example, while it is known to ablate gum tissue by contacting a sphere electrode with gum tissue in a repeated “pecking” motion, the pecking method is slow and imprecise, leading to numerous drawbacks in execution and result.

In some embodiments, at least the working portion 120 of the electrode member 110 is formed of a metal core (e.g., brass or molybdenum) surrounded by a cladding (e.g., a silver alloy). Suitable materials of this type are disclosed in U.S. Published Patent Application No. 2007/0055226, the disclosure of which is incorporated herein by reference. A cladding of silver alloy can provide less surface damage, less pain and suffering, faster healing time, and a smoother brush stroke with less coagulative tissue clinging to the working portion 120 and obscuring the surgical field.

### EXAMPLES

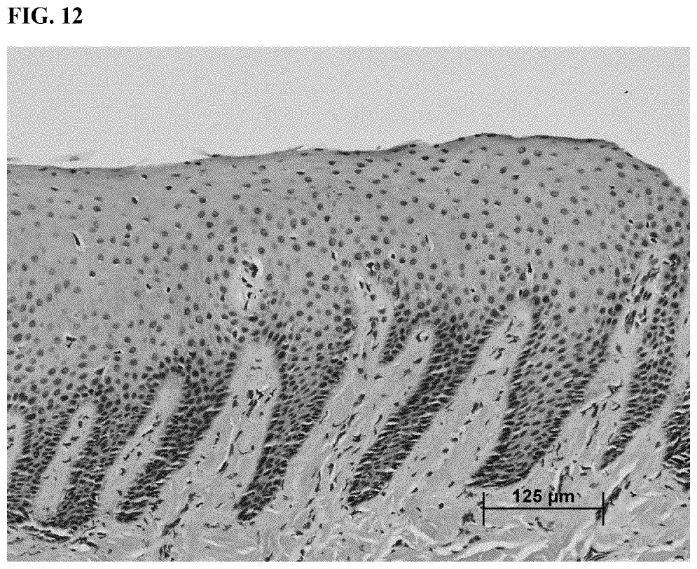

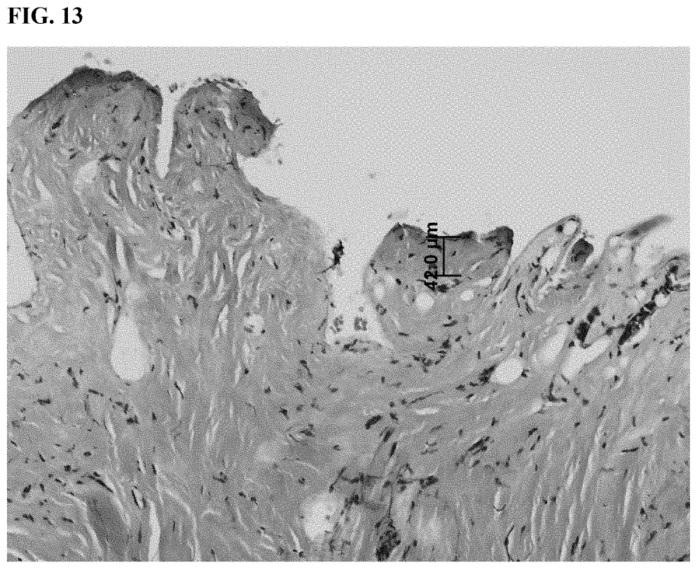

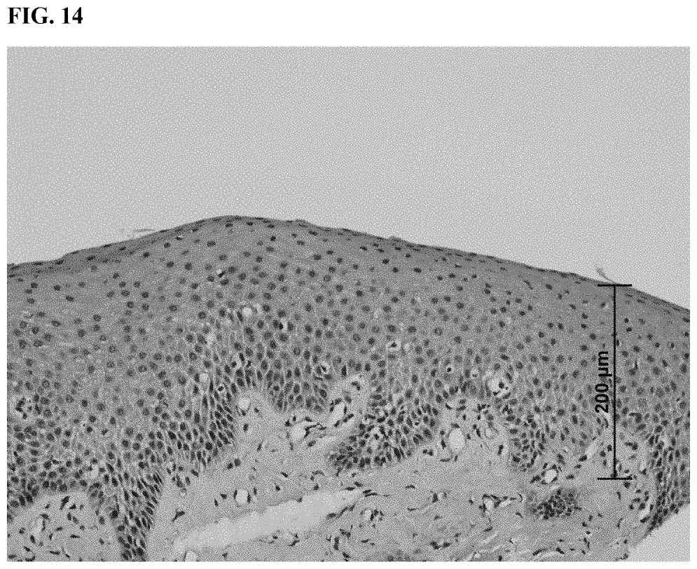

A pigmented tissue was treated according to embodiments of the present invention which included contacting the pigmented tissue with an electrode. The pigmented tissue prior to treatment is shown in FIG. 12. As can be see in FIG. 13, which is a histological image of the tissue on the day the procedure following treatment, all epithelium and pigmentation was removed from the treated tissue. In particular, FIG. 13 shows that the method removed the rete pegs and left the topography of the dermal papillae with minimal damage to a few areas of the dermal papillae. FIG. 14 shows that months after treatment the treated tissue re-epithelized and that the re-epithelized tissue was not pigmented tissue.

Many alterations and modifications may be made by those having ordinary skill in the art, given the benefit of present disclosure, without departing from the spirit and scope of the invention. Therefore, it must be understood that the illustrated embodiments have been set forth only for the purposes of example, and that it should not be taken as limiting the invention as defined by the following claims. The following claims, therefore, are to be read to include not only the combination of elements which are literally set forth but all equivalent elements for performing substantially the same function in substantially the same way to obtain substantially the same result. The claims are thus to be understood to include what is specifically illustrated and described above, what is conceptually equivalent, and also what incorporates the essential idea of the invention.

What is claimed:

1. A method for separating two or more dissimilar materials, the method comprising:
providing an electrode having a contact surface, optionally wherein the contact surface has a curved profile;
placing the contact surface in contact with at least one material of the two or more dissimilar materials; and
sliding the contact surface across and in contact with the at least one material while applying electrosurgical currents via the contact surface to thereby separate the two or more dissimilar materials.

2. The method of claim 1, wherein the two or more dissimilar materials are organic materials.

3. The method of claim 1, wherein the two or more dissimilar materials are two more dissimilar tissues of a subject.

4. The method of claim 1, wherein the subject is a human.

5. The method of claim 1, wherein the subject is an animal (e.g., a dog, cat, horse, etc.).

6. The method of claim 3, wherein the two or more dissimilar tissues comprise epithelium tissue, periosteum tissue, loose connective tissue, dense connective tissue, an oral mucosa, tissue from a portion of the alimentary canal, bone tissue, adipose tissue, glandular tissue, muscle tissue, tendon tissue, dermis tissue, epidermis tissue, subcutaneous tissue, and any combination thereof.

7. The method of claim 3, wherein a first tissue of the two or more dissimilar tissues is a periosteum tissue or an epithelium tissue and a second tissue of the two or more dissimilar tissues is dense connective tissue, loose connective tissue, bone tissue, adipose tissue, and/or glandular tissue.

8. The method of claim 3, wherein a first tissue of the two or more dissimilar tissues is a dermis and a second tissue of the two or more dissimilar tissues is a subcutaneous tissue.

9. The method of claim 3, wherein a first tissue of the two or more dissimilar tissues is a muscle or tendon and a second tissue of the two or more dissimilar tissues is bone tissue.

10. The method of claim 3, wherein at least one of the two or more dissimilar tissues is modified prior to placing the contact surface in contact with the at least one material.

11. The method of claim 10, wherein the at least one of the two or more dissimilar tissues is modified by the subject ingesting a substance (e.g., water) that alters the impedance of at least one tissue.

12. The method of claim 10, wherein at least one of the two or more dissimilar tissues is modified by introducing a substance in and/or on the tissue(s).

13. The method of claim 12, wherein the substance is a conductive (e.g., saline) solution.

14. The method of claim 10, wherein a substance is introduced into an interface between the two or more dissimilar materials.

15. The method of claim 14, wherein the substance is a conductive (e.g., saline) solution.

16. The method of claim 10, wherein the two or more dissimilar materials comprise two or more dissimilar types of polymers.

17. The method of claim 1 wherein the contact surface has a curved profile that has a minimum arc radius of at least 1.5 mm.

18. The method of claim 1 comprising:
providing an electrode set including a plurality of electrodes having contact surfaces of dissimilar widths from one another, optionally wherein each of the electrodes have a curved profile; and
selecting the electrode from the set of electrodes.

19. The method of claim 1 wherein at least one of the two or more dissimilar materials (e.g., tissues) is undamaged by the separation.

20. A method for separating at least two dissimilar materials from an article, the method comprising:
providing an electrode having a contact surface, optionally wherein the contact surface has a curved profile;
placing the contact surface in contact with a surface of an article, the article comprising the at least two dissimilar materials; and
sliding the contact surface across and in contact with the surface of the article while applying electrosurgical currents to the surface of the article via the contact surface to thereby separate the at least two dissimilar materials of the article.
